(19) United States
(12) Patent Application Publication
(10) Pub. No.: US 2023/0227926 A1
(43) Pub. Date: Jul. 20, 2023

(54) ISOTHERMAL REAL-TIME PCR METHOD FOR DETERMINING PRESENCE OF A PRE-DETERMINED NUCLEIC ACID SEQUENCE IN ANIMAL SAMPLES

(71) Applicant: ENDER HOLDING AG, Bern (CH)

(72) Inventors: Samuel ZÜRCHER, Bern (CH); Alexander LÜTHI, Bern (CH); Lea WEIBEL, Bern (CH)

(21) Appl. No.: 18/009,498

(22) PCT Filed: Jun. 9, 2021
(86) PCT No.: PCT/EP2021/065546

§ 371 Date: Dec. 9, 2022

(30) Foreign Application Priority Data
Jun. 9, 2020 (EP) 20179108.4

(51) Int. Cl. C12Q 1/70

(52) U.S. Cl. CPC C12Q 1/701; C12Q 1/705

(57) ABSTRACT

The present invention relates to a method for determining presence of a pre-determined nucleic acid sequence in a sample, the method comprising the steps of adding one or more enzyme(s) providing activities of RNA- and/or DNA-dependent DNA polymerase activity and strand-displacement activity to the sample to be analysed for the presence of the pre-determined nucleic acid sequence; adding at least five DNA primers to the sample to be analysed for the presence of the pre-determined nucleic acid sequence, wherein at least one DNA primer comprises a sequence hybridisable to the nucleic acid sequence and at least one DNA primer comprises a sequence hybridisable to the DNA sequence reverse-complementary to the nucleic acid sequence; incubating the sample resulting at a fixed temperature; determining whether an elongated DNA sequence is present in the sample, wherein presence of the elongated DNA sequence in the sample is indicative of the presence of the pre-determined nucleic acid sequence in the sample, wherein the sample is obtained from an animal and wherein no F3 primer is used.

### RELATED APPLICATIONS

The instant application is a 35 U.S.C. § 371 filing of International Patent Application No. PCT/EP2021/065546, filed Jun. 9, 2021, which claims priority to European Patent Application No. 20179108.4, filed Jun. 9, 2020, the entire contents of which are incorporated herein by reference for all purposes.

### SEQUENCE LISTING

The instant application contains a Sequence Listing which has been submitted electronically in ASCII format and is hereby incorporated by reference in its entirety. Said ASCII copy, created on Dec. 8, 2022, is named 737570_VSO9-007US_ST25.txt and is 5,707 bytes in size.

The present invention relates to a method for determining presence of a pre-determined nucleic acid sequence in a sample, the method comprising the steps of adding one or more enzyme(s) providing activities of RNA- and/or DNA-dependent DNA polymerase activity and strand-displacement activity to the sample to be analysed for the presence of the pre-determined nucleic acid sequence; adding at least five DNA primers to the sample to be analysed for the presence of the pre-determined nucleic acid sequence, wherein at least one DNA primer comprises a sequence hybridisable to the nucleic acid sequence and at least one DNA primer comprises a sequence hybridisable to the DNA sequence reverse-complementary to the nucleic acid sequence; incubating the sample resulting at a fixed temperature; determining whether an elongated DNA sequence is present in the sample, wherein presence of the elongated DNA sequence in the sample is indicative of the presence of the pre-determined nucleic acid sequence in the sample, wherein the sample is obtained from an animal and wherein no F3 primer is used.

Various methods exist for determining whether nucleic acid sequences are present in samples obtained from animals. Most methods rely on nucleic acid molecules hybridisable to a pre-determined sequence, such as for example a sequence expected to be present in a pathogen.

However, existing tests are either highly sensitive and specific but slow or quickly provide a result which lacks appropriate reliability. Thus, there is a need in the art for a method providing a highly sensitive and specific and quick result.

The above technical problem is solved by the embodiments provided herein and as characterized in the claims.

Accordingly, the present invention relates to, inter alia, the following embodiments.
1. A method for determining presence of a pre-determined nucleic acid sequence in a sample, the method comprising the steps of:
   (a) adding one or more enzyme(s) providing activities of RNA- and/or DNA-dependent DNA polymerase activity and strand-displacement activity to the sample to be analysed for the presence of the pre-determined nucleic acid sequence;
   (b) adding at least five DNA primers to the sample to be analysed for the presence of the pre-determined nucleic acid sequence, wherein at least one DNA primer comprises a sequence hybridisable to the nucleic acid sequence and at least one DNA primer comprises a sequence hybridisable to the DNA sequence reverse-complementary to the nucleic acid sequence;
   (c) incubating the sample resulting from steps (a) and (b) at a fixed temperature;
   (d) determining whether an elongated DNA sequence is present in the sample, wherein presence of the elongated DNA sequence in the sample is indicative of the presence of the pre-determined nucleic acid sequence in the sample
   wherein the sample is obtained from an animal.
2. The method of embodiment 1, wherein four of the at least five primers are forward inner primer (FIP), backward inner primer (BIP), loop primer forward (LPF) and loop primer backwards (LPB), respectively.
3. The method of embodiment 1 and 2, wherein the fifth primer is a B3 primer.
4. The method of any one of embodiments 1 to 3, wherein the pre-determined nucleic acid sequence is an RNA or DNA sequence.
5. The method of any one of embodiments 1 to 4, wherein the pre-determined RNA or DNA sequence is comprised in a pathogen.
6. The method of embodiment 5, wherein the pathogen is a virus, a bacterium, a fungus or a parasite.
7. The method of embodiment 6, wherein the pathogen is Streptococcus equi equi, Equine herpes virus, in particular Equine herpes virus 1 or 4, or Equine influenza virus.
8. The method of any one of embodiments 1 to 7, wherein the fixed temperature is between 50 and 75° C.
9. The method of any one of embodiments 1 to 8, wherein the sample in step (c) is incubated for 1 to 120 minutes.
10. The method of any one of embodiments 1 to 9, wherein presence of the double-stranded elongated DNA sequence in the sample is determined by using a nucleic acid molecule hybridisable to the double-stranded elongated DNA sequence, in particular wherein the nucleic acid molecule is labelled, using a molecule that intercalates in the double-stranded elongated DNA sequence or using turbidity measurement.
11. An anti-infective composition for use in the treatment of an infection of a pathogen, wherein the animal has previously been determined to be infected by the pathogen using the method of any one of embodiments 1 to 10.
12. The anti-infective composition for use of embodiment 11, wherein the animal is a horse.
13. The anti-infective composition for use of embodiment 11 or 12, wherein the pathogen is a virus, a bacterium, a fungus or a parasite.
14. The anti-infective composition for use of embodiments 11 or 13, wherein the anti-infective composition comprises an antiviral, antibiotic, antifungal or antiparasitic drug, respectively.
15. The anti-infective composition of embodiments 12 to 14, wherein the pathogen is Streptococcus equi equi, Equine herpes virus, in particular Equine herpes virus 1 or 4, or Equine influenza virus.

Accordingly, in a first aspect, the invention relates to a method for determining presence of a pre-determined nucleic acid sequence in a sample, the method comprising the steps of adding one or more enzyme(s) providing activities of RNA- and/or DNA-dependent DNA polymerase activity and strand-displacement activity to the sample to be analysed for the presence of the pre-determined nucleic acid sequence; adding at least five DNA primers to the sample to be analysed for the presence of the pre-determined nucleic acid sequence, wherein at least one DNA primer comprises a sequence hybridisable to the nucleic acid sequence and at least one DNA primer comprises a sequence hybridisable to the DNA sequence reverse-complementary to the nucleic acid sequence; incubating the sample resulting at a fixed temperature; determining whether a double-stranded elongated DNA sequence is present in the sample, wherein presence of the double-stranded elongated DNA sequence in the sample is indicative of the presence of the pre-determined nucleic acid sequence in the sample, wherein the sample is obtained from an animal and wherein no F3 primer is used.

The term “pre-determined nucleic acid sequence”, as used herein, refers to a nucleic acid sequence, preferably an RNA or DNA sequence, where the skilled person is aware that it may be comprised in a sample obtained from an animal (e.g. tissue sample, bronchoalveolar lavage, bronchial wash, pharyngeal exudate, tracheal aspirate, blood, serum, plasma, bone, skin, soft tissue, intestinal tract specimen, genital tract specimen, breast milk, lymph, cerebrospinal fluid, pleural fluid, sputum, urine, a nasal secretion, tears, bile, ascites fluid, pus, synovial fluid, vitreous fluid, vaginal secretion, semen and/or urethral tissue). In particular, the pre-determined nucleic acid sequence, within the present invention, is a sequence that is detectable using the method of the present invention. That is, a nucleic acid sequence available to the skilled person is pre-determined if the skilled person can determine whether the sequence will likely be detectable in a sample obtained from an animal using the methods as provided herein. Within the present invention, the pre-determined nucleic acid sequence comprises at least one primer binding site that is at least partially identical to at least one of the primers used in the methods of the invention. Primer binding sites are considered identical to a primer site if the sequence is exactly identical or if they differ only in that one sequence comprises uracil instead of thymidine and/or if they differ only in that one sequence comprises one or more modified nucleotides instead of the respective non-modified nucleotide(s).

The terms “DNA primer” or “primer”, as used herein, refer to a nucleic acid molecule comprising a 3′-terminal —OH group that, upon hybridisation to a complementary nucleic acid sequence, can be elongated, e.g., via an enzymatic nucleic acid replication reaction. The primer set according to the present invention is used for amplification of nucleic acids, that is, for a LAMP analysis or a RT-LAMP analysis. Both the upper and lower limits of the length of the primer are empirically determined. The primer described herein can be a forward primer or a reverse primer. The term “backward primer”, as used herein, refers to a primer priming the antisense strand of a DNA sequence to allow the polymerase to extend in one direction along the complementary strand of a DNA sequence. At least one backward primer also serves as the RT primer for reverse transcription. The term “forward primer”, as used herein, refers to a primer priming the sense strand of a DNA sequence to allow a polymerase to extend in one direction along one strand of a DNA sequence.

An enzyme providing activities of RNA- and/or DNA-dependent DNA polymerase activity can synthesize DNA in the 5′->3′ direction based on a template composed of a DNA or RNA strand. As the skilled person is aware, such an enzyme will be successively adding nucleotides to the free 3′-hydroxyl group of the template. In this regard, the template strand determines the sequence of the added nucleotides based on Watson-Crick base pairing. The activity of the DNA polymerase may be RNA- and/or DNA-dependent. Exemplary polymerases include, but are not limited to Bst DNA polymerase, Vent DNA polymerase, Vent (exo-) DNA polymerase, Deep Vent DNA polymerase, Deep Vent (exo-) DNA polymerase, Bca (exo-) DNA polymerase, DNA polymerase I Klenow fragment, Φ29 phage DNA polymerase, Z-Taq™ DNA polymerase, ThermoPhi polymerase, 9° Nm DNA polymerase, and KOD DNA polymerase. See, e.g., U.S. Pat. Nos. 5,814,506; 5,210,036; 5,500,363; 5,352,778; and 5,834,285; Nishioka, M., et al. (2001) J. Biotechnol. 88, 141; Takagi, M., et al. (1997) Appl. Environ. Microbiol. 63, 4504.

As an enzyme providing activities of RNA-dependent DNA polymerase activity any suitable reverse transcriptase may be employed. In this regard, the enzyme to be used is not particularly limited, with the proviso that it has the activity to synthesize cDNA using RNA as the template. In addition, a substance which improves heat resistance of the nucleic acid amplification enzyme, such as trehalose, can be added.

When simply expressed as “5′-end side” or “3′-end side” in this specification, it means the direction in the chain which is regarded as the template in all cases. Also, when described that the 3′-end side becomes the starting point of complementary chain synthesis, it means that the 3′-end side —OH group is the starting point of complementary chain synthesis.

The term “strand displacement”, as used herein, refers to the ability of an enzyme to separate the DNA and/or RNA strands in a double-stranded DNA molecule and/or in a double-stranded RNA molecule during primer-initiated synthesis.

The term “hybridisation”, as used herein, refers to the annealing of complementary nucleic acid molecules. When two nucleic acids “hybridise to” each other, or when one nucleic acid “hybridises to” another, the two nucleic acid molecules exhibit a sufficient number of complementary nucleobases that the two nucleic acid molecules can anneal to each other under the particular conditions (e.g., temperature, salt and other buffer conditions) being utilized for a particular reaction. The most common mechanism of hybridisation involves hydrogen bonding (e.g., Watson-Crick, Hoogsteen or reversed Hoogsteen hydrogen bonding) between complementary nucleobases of the nucleic acid molecules. Hybridisation can occur under varying conditions. Stringent conditions are sequence-dependent and are determined by the nature and composition of the nucleic acid molecules to be hybridised. Nucleic acid hybridisation techniques and conditions are known to the skilled artisan and have been described extensively. See, e.g., Sambrook et al, Molecular Cloning: A Laboratory Manual 2nd ed. Cold Spring Harbor Press, 1989; Ausubel et al, 1987, Current Protocols in Molecular Biology; Greene Publishing and Wiley-Interscience, New York; Tijessen, 1993, Hybridization with Nucleic Acid Probes, Elsevier Science Publishers, B.V.; and Kricka, 1992, Non-Isotopic DNA Probe Techniques, Academic Press, San Diego, Calif.

The term “F3”, as used herein, refers to the outer forward primer of a primer set.

Within the present invention, it was surprisingly found that a five-primer system, wherein the F3 primer is omitted, is most efficient in detecting a pre-determined nucleic acid sequence. “Most efficient” as used herein means that detection is as fast and sensitive as commonly used techniques but maintains reliability, which is a prerequisite in tests used for detecting nucleic acids such as nucleic acids derived from animal samples. In addition, it was found that by using five primers instead of six primers as in the standard LAMP technology, shorter target sequences can be detected.

It is preferred within the methods of the present invention that five DNA primers are used.

In some embodiments, the at least two of the primers employed in the invention are loop primers.

The term “loop primer”, as used herein, refers to a DNA primer comprising a sequence that is hybridisable to at least one loop region of an amplification product of the pre-determined RNA sequence. The loop region is formed by the annealing of a strand of an amplification product to itself. Typically, loop primers hybridise to generated DNA sequences and provide an increased number of starting points for the initiation of further DNA elongation processes. The use of loop primer can accelerate the amplification process.

Within the present invention, it is preferred that the at least five primers comprise a forward inner primer (FIP), backward inner primer (BIP), loop primer forward (LPF) and loop primer backwards (LPB), respectively.

The term “FIP” or “forward inner primer”, as used herein, refers to a forward primer that comprises a sequence for strand initiation and a sequence hybridisable to the same FIP-initiated strand.

The term “BIP” or “backward inner primer”, as used herein, refers to a backward primer that comprises a sequence for strand initiation and a sequence hybridisable to the same BIP-initiated strand.

The term “loop primer forward” or “LPF”, as used herein, refers to a loop primer that is a forward primer.

The term “loop primer backwards” or “LPB”, as used herein, refers to a loop primer that is a backwards primer.

Preferably, the at least five primers further comprise a B3 primer.

The term “B3”, as used herein, refers to the outer backward primer of a primer set.

The DNA Primer described herein that specifically binds to a target nucleic acid or its complementary sequence may be at least 10, 15, or 16 nucleotides in length, at least 10, 14, 15 or 16 nucleotides for B3, at least 25, 30, 33, or 36 nucleotides for FIP and BIP, and at least 10, 15, 17, or 18 for LPF and LPB. DNA Primers that specifically bind to a target nucleic acid sequence may have a nucleic acid sequence at least 80% complementarity, particularly 90% complementarity, more particularly 95%, 96%, 97%, 98%, 99% or 100% complementarity with the corresponding region.

These terms are commonly used in methods related to loop-mediated isothermal amplification (LAMP) methods, such as those described by Nagamine et al. 2002. Molecular and Cellular Probes 16. 223-229.

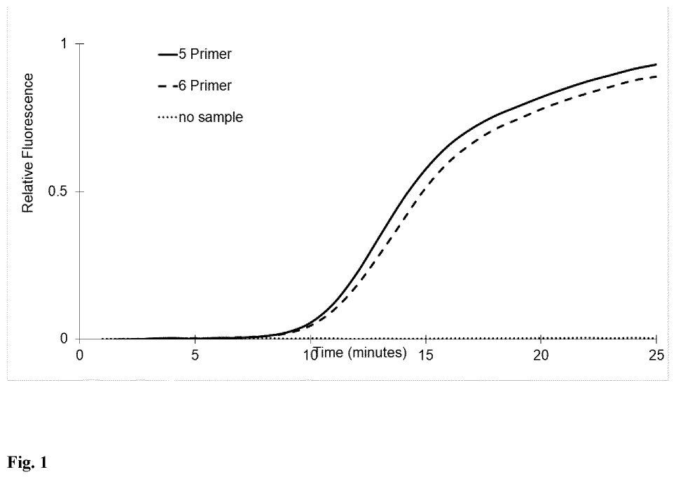

Within the methods of the present invention no F3 primer is used and it is thus preferred that the fifth primer is a B3 primer. This is because it was surprisingly found by the inventors that in the presence of a B3 primer but absence of an F3 primer, detection is as fast and as sensitive. Using the methods of the present invention, detection was observed to be possible within ten minutes and as sensitive to detect a low number of pre-determined nucleic acid sequence in a sample. That is, as it is shown in the appended Examples, a positive detection of a pre-determined nucleic acid sequence was achieved using five primers, in particular FIP, BIP, LPF, LPB and B3, within ten minutes after addition of primers and enzymes (e.g., FIG. 1.). Accordingly, the methods of the present invention, for the first time, provide a reliable and fast way to detect infections which is important in controlling outbreak of the same.

The use of less than 6 primers is preferred within the methods of the present invention. The use of less primers requires less primer binding sites and thus shorter target sequences can be detected. This is particularly important since the availability of target sequence, in particular conserved and target specific sequences, is highly limited. In this respect, it was now surprisingly found that the specific methods as provided herein provide the same sensitivity when using 5 primers instead of 6 primers. This surprising demonstration has the further advantage in cases where more than one primer system is used in the same method, e.g. for detecting more than one target sequence in the same or different pathogen.

Within the methods of the present invention, one or more enzyme(s) providing activities of RNA- and/or DNA-dependent DNA polymerase activity and strand-displacement activity are used. That is, in case of an RNA sequence, all three activities are to be added to the RNA sequence to be analyzed. In case of a DNA sequence, activity of the RNA-dependent DNA polymerase is not required. The activities can be provided by one enzyme having all two/three activities, or several enzymes each having one or more of the two/three activities.

It is preferred that the pre-determined nucleic acid sequence is an RNA or DNA sequence.

In some embodiments, it is preferred that the pre-determined RNA or DNA sequence is comprised in a pathogen. That is, the method provided herein is used to detect presence of a nucleic acid sequence of a pathogen in a sample obtained from an animal. The invention thus relates to, inter alia, a method for diagnosing whether an animal suffers or is likely to suffer from a disease caused by a pathogen, wherein presence of a nucleic acid sequence of said pathogen was determined using the methods provided herein.

It is preferred that the animal is a mammal, preferably a horse. The animal is not a human.

The term “horse”, as used herein, refers to an animal of the genus Equus, preferably of the species Equus ferus “Horse” includes all breeds, including, without limitation, Appaloosa, Noriker, Knabstrupper, American Miniature, Pony of the America, Australian spotted pony, British spotted pony, Altai, Mongolian Pony, Colorado Ranger Horse, Falabella, Spanish Mustang, and Karabaier or any other LP carrying breed.

Diagnosing of diseases in animals and sample acquisition form animals is particularly challenging due to limitations in cooperation and/or communication. Furthermore, the spread of pathogens may be difficult to limit depending on animal housing conditions. Therefore, the efficient detection of pre-determined nucleic acid sequences in samples obtained from animals is particularly useful in animals such as horses.

In some embodiments, the pathogen is a virus, a bacterium, a fungus or a parasite.

The term “virus”, as used herein, refers to an infectious agent that replicates only inside the living cells of an organism. Any nucleic acid comprising stadium (e.g. inside the cell or in the virus envelope) may be determined by the invention. In some embodiments, the method of the invention is used to determining the presence of a pre-determined nucleic acid sequence of at least one virus of the genus selected from the group consisting of African horse sickness virus, Bovine papillomavirus, Equid alphaherpesvirus 1, Equid alphaherpesvirus 3, Equid gammaherpesvirus 2, Equid gammaherpesvirus 5, Equine encephalosis virus, Equine influenza virus, Equine viral arteritis, Erbovirus, Henipavirus, Togavirus, Venezuelan equineencephalitis virus, Western equine encephalitis virus, Equine infectious anemia virus, West nile virus.

In some embodiments, the method of the invention is used to determining the presence of a pre-determined nucleic acid sequence of at least one bacteria from the genus selected from the group consisting of Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus Alteromonas Amycolata, Amycolatopsis, Anaerobospirillum, Anaerorhabdus, “Anguillina”, Arachnia, Arcanobacterium, Arcobacter, Arthrobacter, Atopobium, Aureobacterium, Bacillus, Bacteroides, Balneatrix, Bartonella, Bergeyella, Bifidobacterium, Bilophila, Branhamella, Borrelia, Bordetella, Brachyspira, Brevibacillus, Brevibacterium, Brevundimonas, Brucella, Burkholderia, Buttiauxella, Butyrivibrio, Calymmatobacterium, Campylobacter, Capnocytophaga, Cardiobacterium, Catonella, Cedecea, Cellulomonas, Centipeda, Chlamydia, Chlamydophila, Chromobacterium, Chyseobacterium, Chryseomonas, Citrobacter, Clostridium, Collinsella, Comamonas, Corynebacterium, Coxiella, Cryptobacterium, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Flexispira, Francisella, Fusobacterium, Gardnerella, Gemella Globicatella, Gordona, Haemophilus, Hafnia, Helicobacter, Helococcus, Holdemania, Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptotrichia, Leuconostoc, Listeria, Listonella, Megasphaera, Methylobacterium, Microbacterium, Micrococcus, Mitsuokella, Mobiluncus, Moellerella, Moraxella, Morganella, Mycobacterium, Mycoplasma, Myroides, Neisseria, Nocardia, Nocardiopsis, Ochrobactrum, Oeskovia Oligella, Orientia, Paenibacillus, Pantoea, Parachlamydia, Pasteurella, Pediococcus, Peptococcus, Peptostreptococcus, Photobacterium, Photorhabdus, Plesiomonas Porphyrimonas, Prevotella, Propionibacterium, Proteus, Providencia, Pseudomonas, Pseudonocardia, Pseudoramibacter, Psychrobacter, Rahnella, Ralstonia, Rhodococcus, Rickettsia, Rochalimaea, Roseomonas, Rothia, Ruminococcus, Salmonella, Selenomonas, Serpulina, Serratia, Shewenella, Shigella, Simkania, Slackia, Sphingobacterium, Sphingomonas, Spirillum, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Tropheryma, Tsakamurella, Turicella, Ureaplasma, Vagococcus, Veillonella, Vibrio, Weeksella, Wolinella, Xanthomonas, Xenorhabdus, Yersinia and Yokenella.

In some embodiments, the method of the invention is used to determining the presence of a pre-determined nucleic acid sequence of at least one fungus selected from the group consisting of Malassezia furfur, Malassezia slooffiae, Malassezia obtusa, Malasseziaglobosa, Malassezia restricta, Malassezia pachydermatis, Fonsecaea spp., Microsporum canis, Microsporum gypseum, Trichophyton equinum, Trichophyton mentagrophytes, Geotrichum candidum, Aspergillus spp., Candida spp., Scopulariopsis brevicaulis, Scedosporium spp. Trichophyton spp., Histoplasma capsulatum var. Capsulatum, Histoplasma capsulatum var. farciminosum, Scedosporium/Pseudallescheria complex, Madurella mycetomatis, Curvularia verruculosa, Phialophoraoxyspora, Scedosporium/Pseudallescheria complex, Alternaria spp., Drechslera spicifera, Curvularia spp., Pythium isidiosum, Sporothrix schenckii complex, Conidiobolus coronatus (i.e., Entomophthora coronata), Conidiobolus lamprauges, Basidiobolushaptosporus haptosporus, Rhizopus stolonifer, Absidia corimbifera, Mucor spp., Emmonsia, Blastomyces dermatitidis, Coccidioides immitis, Cryptococcus gattii, Cryptococcus neoformans, Pneumocistis sp.

As used herein, “parasite” refers to an organism that lives in or on a second organism. In some embodiments, the method of the invention is used to determining the presence of a pre-determined nucleic acid sequence of at least one parasite from the genus selected from the group consisting of Ectoparasites, Protozoan organisms and/or Helminths such as Tapeworms, Flukes and/or Roundworms. In some embodiments, the method of the invention is used to determining the presence of a pre-determined nucleic acid sequence of at least one parasite selected from the group consisting of Strongylus vulgaris, Cyathostomin spp., Parascaris equorum, Oxyuris equi, Strongyloides Westeri, Anoplocephala spp., Dictyocaulus arnfieldi, Gastrophilus.

It is particular preferred that the pathogen is Streptococcus equi equi, Equine herpes virus, in particular Equine herpes virus 1 or 4, or Equine influenza virus.

In the methods of the present invention, in particular in step (c) thereof, the temperature can be fixed.

The term “fixed temperature”, as used herein, refers to keeping the temperature condition constant or almost constant so that enzymes and primers can substantially function. The almost constant temperature condition means that not only the set temperature is accurately maintained but also a slight change in the temperature is acceptable within such a degree that it does not spoil substantial functions of the enzymes and primers. For example, a change in temperature of approximately from 0 to 10° C. is acceptable.

The nucleic acid amplification reaction under a fixed temperature can be carried out by keeping the temperature at such a level that activity of the enzyme to be used can be maintained. In addition, in order to effect annealing of a primer with the target nucleic acid in said nucleic acid amplification reaction, for example, to set the reaction temperature may be set to the temperature of around the Tm value of the primer or lower than that, and it is preferred to set it at a level of stringency by taking the Tm value of the primer into consideration. In said nucleic acid amplification reaction, the amplification reaction can be repeated until the enzyme is inactivated or one of the reagents including primers is used up.

That is, the one or more enzyme(s), DNA primers and the sample to be analyzed are incubated in the same tube at a constant temperature. The temperature is preferably between 50 and 75° C. However, the temperature may also be lower, for example between 30 and 75° C. In an alternative embodiment, a touchdown temperature step is used. That is, the temperature is lowered during the course of the analysis, for example starting at a temperature of 70° C. that is subsequently lowered to 50° C.

In the methods of the present invention, the one or more enzyme(s), DNA primers and the sample to be analyzed are incubated in the same tube for a time between 1 and 120 minutes, preferably between 1 and 60, 1 and 45, 1 and 30 or between 1 and 15 minutes. In a preferred embodiment, the sample is incubated for 3, 4, 5, 6, 7, 8, 9, 10, 11, 12, 13, 14, 15, 16, 17, 18, 19, 20, 21, 22, 23, 24, 25, 26, 27, 28, 29 or 30 minutes.

Within the present invention, it is in one embodiment preferred that the virus is an Equine influenza virus. In such an embodiment of the invention, the preferred primer sequences are shown below:

(a) FIP primer sequence:
(SEQ ID NO: 1)
CAA GTC TCT GCG CGA TCT AGG TCG AAA CGT ACG TTC;
and/or
(b) BIP primer sequence:
(SEQ ID NO: 2)
GAA GAT GTC TTT GCA GGG TTG GTC TTG TCT TTA GCC;
and/or
(c) LPF primer sequence:
(SEQ ID NO: 3)
TTT GAG GGG GCC TGA TGG;
and/or
(d) LPB primer sequence:
(SEQ ID NO: 4)
ACC GAT CTT GAG GCA CTC;
and/or
(e) B3 primer sequence:
(SEQ ID NO: 5)
AAT CCC TTT AGT YAG AG.

The above primer sequences target a sequence of Equine influenza virus RNA. In particular, the above primer target the following sequence

#JN85 0794.1_Influenza_A_virus_(A/equine/Newmarket/11/2003
(H3N8))_segment_7_matrix_protein_2_(M2)_and_matrix_protein_1_(M1)_
genes_compl ete_cds
(SEQ ID NO: 13)
ATGAGTCTTCTGACCGAGGTCGAAACGTACGTTCTCTCTATCGTACCATCAGGCC
CCCTCAAAGCCGAGATCGCGCAGAGACTTGAAGATGTCTTTGCAGGGAAGAACA
CCGATCTTGAGGCACTCATGGAATGGCTAAAGACAAGACCAATCCTGTCACCTTT
GACTAAAGGGATTTTAGGATTTGTATTCACGCTCACCGTGCCCAGTGAGCAAGGA
CTGCAGCGTAGACGCTTTGTCCAAAATGCCCTTAGTGGAAACGGAGATCCAAACA
ACATGGACAGAGCAGTAAAACTGTACAGGAAGCTTAAAAGAGAAATAACATTCC
ATGGGGCAAAAGAGGTGGCACTAAGCTATTCCACTGGTGCACTAGCCAGCTGCAT
GGGACTCATATACAACAGAATGGGAACTGTGACAACCGAAGTGGCATTTGGCCT
GGTATGCGCCACATGTGAACAGATTGCTGATTCCCAGCATCGATCTCACAGGCAG
ATGGTGACAACAACCAACCCATTAATCAGACATGAAAACAGAATGGTATTAGCC
AGTACCACGGCTAAAGCCATGGAACAGATGGCAGGATCAAGTGAGCAGGCAGCA
GAGGCCATGGAGGTTGCTAGTAAGGCTAGGCAGATGGTACAGGCAATGAGAACC
ATTGGGACCCACCCTAGCTCCAGTGCCGGTTTGAAAGATGATCTCCTTGAAAATT
TACAGGCCTACCAGAAACGGATGGGAGTGCAAATGCAGCGATTCAAGTGATCCT
CTCGTTATTGCAGCAAGTATCATTGGGATCTTGCACTTGATATTGTGGATTCTTGA
TCGTCTTTTCTTCAAATTCATTTATCGTCGCCTTAAATACGGGTTGAAAAGAGGGC
CTTCTACGGAAGGAGTACCTGAGTCTATGAGGGAAGAATATCGGCAGGAACAGC
AGAATGCTGTGGATGTTGACGATGGTCATTTTGTCAACATAGAGCTGGAGTAA

In some embodiments, the primers used in the methods of the invention, in particular for Equine influenza, comprise at least one selected from the group of:

a) a FIP primer comprising a sequence that has at least 88%, 91%, 94%, 97% or 100% sequence identity to the sequence: CAA GTC TCT GCG CGA TCT AGG TCG AAA CGT ACG TTC (SEQ ID NO: 1), which sequence still provides the primer functionality,

b) a BIP primer comprising a sequence that has at least 88%, 91%, 94%, 97% or 100% sequence identity to the sequence: GAA GAT GTC TTT GCA GGG TTG GTC TTG TCT TTA GCC (SEQ ID NO: 2), which sequence still provides the primer functionality,

c) a LPF primer comprising a sequence that has at least 83%, 88%, 94%, or 100% sequence identity to the sequence TTT GAG GGG GCC TGA TGG (SEQ ID NO: 3), which sequence still provides the primer functionality,

d) a LPB primer comprising a sequence that has at least 83%, 88%, 94%, or 100% sequence identity to the sequence: ACC GAT CTT GAG GCA CTC (SEQ ID NO: 4), which sequence still provides the primer functionality, and

e) a B3 primer comprising a sequence that has at least 82%, 88%, 94% or 100% sequence identity to the sequence: AAT CCC TTT AGT YAG AG (SEQ ID NO: 5), which sequence still provides the primer functionality,

preferably wherein the primer functionality is primer functionality at the SEQ ID NO: 13.

Within the present invention, it is in one embodiment preferred that the virus is an Equine herpes virus type 1. In such an embodiment of the invention, the preferred primer sequences are shown below:

(a) FIP primer sequence:
(SEQ ID NO: 7)
GTC GTA RAA CCT GAG AGC GGC CTG CTA GAC TAC AGC;
and/or
(b) BIP primer sequence:
(SEQ ID NO: 8)
CGA CAG CGT GGT CAA CGT TGA AAA AGC TGG CGA TCC;
and/or
(c) LPF primer sequence:
(SEQ ID NO: 9)
GAG CTG GTT GCG GCG CTG;
and/or
(d) LPB primer sequence:
(SEQ ID NO: 10)
ATA CCG CAG TGA TTA TGC;
and/or
(e) B3 primer sequence:
(SEQ ID NO: 11)
TCC CCC ACT TTA CCC AG.

The above primer sequences target a sequence of Equine herpesvirus type 1. In particular, the above primer target the following sequence:

#JN705797.1_Equid_herpesvirus_1_strain_TR04_
glycoprotein_B_(glyB)_gene_partial_cds
(SEQ ID NO: 14)
GACACCACGCGAAGACGCAGGTCTCTCCTGTCAGTACCAGAACCTCAGC
CAACCCAAGATGGTGTGCATAGAGAACAAATTCTACATCGCTTGCACAA
ACGAGCAGTGGAGGCAACGGCAGGTACCGATTCTTCCAACGTCACCGCC
AAACAGCTGGAGCTCATCAAAACCACGTCGTCTATCGAGTTTGCCATGC
TACAGTTTGCATACGATCACATCCAATCCCACGTCAATGAAATGCTAAG
TAGAATAGCAACTGCGTGGTGTACCCTCCAAAACAAAGAGCGGACCCTA
TGGAACGAAATGGTGAAGATTAACCCGAGCGCCATAGTCTCCGCAACCC
TTGACGAGCGAGTTGCAGCGAGGGTCCTGGGGGACGTGATAGCTATAAC
GCACTGCGCCAAAATAGAGGGCAACGTGTACTTGCAAAACTCCATGCGC
TCGATGGACAGTAACACGTGCTACTCCCGCCCCCCCGTAACATTTACAA
TTACTAAGAATGCAAACAACAGAGGGTCGATAGAAGGCCAGCTGGGAGA
GGAGAACGAGATTTTCACGGAGCGCAAGCTGATCGAGCCGTGCGCCCTC
AATCAGAAGCGCTACTTTAAGTTTGGCAAAGAGTACGTTTACTACGAGA
ACTACACGTTCGTCCGCAAAGTGCCCCCCACGGAAATCGAGGTTATCAG
CACGTACGTTGAACTAAACTTGACCCTTTTGGAAGACCGCGAGTTTCTG
CCCCTGGAGGTGTACACGCGGGCTGAGCTGGAGGACACCGGCCTGCTAG
ACTACAGCGAAATACAGCGCCGCAACCAGCTCCACGCTCTCAGGTTTTA
TGACATCGACAGCGTGGTCAACGTGGACAATACCGCAGTGATTATGCAG
GGGATCGCCAGCTTTTTCAAGGGCCTGGGTAAAGTGGGGGAGGCCGTGG
GAACGCTCGTTCTCGGCGCCGCCGGCGCTGTTGTTTCAACCGT

In some embodiments, the primers used in the methods of the invention, in particular for Equine herpes virus type 1, comprise at least one selected from the group of:

a) a FIP primer comprising a sequence that has at least 88%, 91%, 94%, 97% or 100% sequence identity to the sequence: GTC GTA RAA CCT GAG AGC GGC CTG CTA GAC TAC AGC (SEQ ID NO: 7), which sequence still provides the primer functionality,

b) a BIP primer comprising a sequence that has at least 88%, 91%, 94%, 97% or 100% sequence identity to the sequence: CGA CAG CGT GGT CAA CGT TGA AAA AGC TGG CGA TCC (SEQ ID NO: 8), which sequence still provides the primer functionality,

c) a LPF primer comprising a sequence that has at least 83%, 88%, 94%, or 100% identical to the sequence GAG CTG GTT GCG GCG CTG (SEQ ID NO: 9), which sequence still provides the primer functionality,

d) a LPB primer comprising a sequence that has at least 83%, 88%, 94%, or 100% sequence identity to the sequence: ATA CCG CAG TGA TTA TGC (SEQ ID NO: 10), which sequence still provides the primer functionality, and

e) a B3 primer comprising a sequence that has at least 82%, 88%, 94% or 100% sequence identity to the sequence: TCC CCC ACT TTA CCC AG (SEQ ID NO: 11), preferably wherein the primer functionality is primer functionality at the SEQ ID NO: 14.

“Percent (%) sequence identity” with respect to a reference sequence is defined as the percentage of nucleotides in a candidate sequence that are identical with the nucleotides in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

However, the skilled person is well-aware how to design alternative or further primer sequences depending on the target sequence to be detected in the sample (see e.g., Jia, B., et al., 2019, Frontiers in microbiology, 10, 2860).

The methods of the present invention comprise a step of determining whether a double-stranded elongated DNA sequence is present in the sample, in particular wherein presence of the double-stranded elongated DNA sequence in the sample is indicative of the presence of the pre-determined nucleic acid sequence in the sample. The skilled person is well-aware of methods suitable to be used for determining presence of a double-stranded DNA sequence in a sample, in particular where the sequence to be detected is known. Thus, any method known to the skilled person for that purpose may be used within the present invention. However, it is preferred that the presence of the elongated double-stranded DNA is determined by using a nucleic acid molecule hybridisable to the elongated double-stranded DNA sequence, in particular wherein the nucleic acid molecule is labelled, using a molecule that intercalates in the elongated double-stranded DNA sequence or using turbidity measurement.

The term “label” or grammatical variations thereof, as used herein, refer to any detectable or signal-generating molecule or reporter molecule. Convenient labels include colorimetric, chemiluminescent, chromogenic, radioactive and fluorescent labels, but enzymatic (e.g. colorimetric, luminescent, chromogenic) or antibody-based labelling methods or signal-generating systems may also be used. Thus, the term “label” as used herein includes not only directly detectable signal-giving or passive moieties, but also any moiety which generates a signal or takes part in a signal generating reaction or that may be detected indirectly in some way. “labelled” as used herein, refers to being connected with or linked to a detectable label. Determining whether an elongated double-stranded DNA sequence is present in the sample may be achieved via fluorescence reporting. The majority of such approaches are based on the use of intercalating dyes, such as ethidium bromide, SYBR Green, EvaGreen and YO-PRO-I (Zhang X, et al. 2013, PLoS One 8(12):e82841; Mair G. et al. 2013, BMC Veterinary Research 9: 108.). As used herein, an agent or dye that “intercalates” refers to an agent or moiety capable of non-covalent insertion between stacked base pairs in a nucleic acid double helix. Determining whether an elongated double-stranded DNA sequence is present in the sample may be achieved by a Fluorescence technique that relies on the mechanism of Forster resonance energy transfer (FRET) (Chen Q, et al., 1997, Biochemistry 36(15):4701-11). In certain embodiments of the invention, the LPB ant/or LPF are labelled at the 5′ end with at least one label and/or acceptor fluorophore.

The term “turbidity”, as used herein, refers to a measure of the suspended and/or soluble particles in a fluid or transparent solid that causes light to be scattered or absorbed. In certain embodiments of the invention, indirect determination of whether an elongated double-stranded DNA sequence is present in the sample relies essentially on the formation of pyrophosphate as a reaction byproduct. Pyrophosphate ions can be released by incorporation of deoxynucleotide triphosphates (dNTPs) into the DNA strand during nucleic acid polymerization and these ions react with divalent metal ions, particularly magnesium ions, present in the reaction mix to produce a white, insoluble magnesium pyrophosphate precipitate as described by Mori Y., et al. 2001 (Biochem. Biophys. Res. Commun. 289: 150-154). This participate results in a progressive increase in the turbidity of the reaction solution and pyrophosphate precipitates can be measured quantitatively in terms of turbidity or observed by the naked eye as a pellet after centrifugation. In an alternative embodiment of the invention, determining whether an elongated double-stranded DNA sequence is present in a sample is achieved through the incorporation of manganese ions and calcein in the reaction, Calcein's fluorescence is naturally quenched by binding of manganese ions. Pyrophosphate production as a reaction byproduct removes manganese ions form the buffer through precipitation, and the increased turbidity coupled with restored calcein fluorescence enables an easy visual read-out upon excitation with either visible or UV light (Tomita N., et al. 2008. Nat. Protoc. 3:877-882). In still another embodiment of the invention, the enzymatic conversion of pyrophosphate into ATP, which is produced during DNA synthesis, is monitored through the bioluminescence generated by thermostable firefly luciferase for determining whether an elongated double-stranded DNA sequence is present in the sample (Gandelman O A., et al. 2010. PLoS One 5(11): e14155). Generally, all methods described by Becherer, Lisa, et al. (“Loop-mediated isothermal amplification (LAMP)—review and classification of methods for sequence-specific detection.” Analytical Methods 12.6 (2020): 717-746) can be combined with the method of the invention.

In a further embodiment, the present invention relates to a method of treating an animal infected by a pathogen, the method comprising administering to the animal an efficient amount of a therapeutic drug, wherein the animal has previously been determined to be infected by the pathogen using the method of the present invention.

In a further embodiment, the present invention relates to an anti-infective composition for use in the treatment of an infection of a pathogen, wherein the animal has previously been determined to be infected by the pathogen using the method of the invention.

In a preferred embodiment, the pathogen is a virus, a bacterium, a fungus or a parasite. In a further preferred embodiment, the therapeutic drug is an antiviral, antibiotic, antifungal or antiparasitic drug, respectively.

The term “antiviral drug”, as used herein, refers to a drug with properties useful in the treatment against a virus-related disease. An antiviral drug may have, inter alia, properties of preventing, inhibiting, suppressing, reducing, adversely impacting, and/or interfering with the growth, survival, replication, function, and/or dissemination of a virus.

In some embodiments, the antiviral drug described herein comprises at least one agent selected from the group vidarabine, acyclovir, penciclovir, ribavirin, zidovudine, amantadine and famciclovir.

The term “antibiotic drug”, as used herein, refers to an agent or a composition with properties useful against bacteria and/or in the treatment of bacteria-related disease. The antibiotic drug may have, inter alia, properties of preventing, inhibiting, suppressing, reducing, adversely impacting, and/or interfering with the growth, survival, replication, function, and/or dissemination of at least one bacterium. In some embodiments, the antibiotic drug described herein comprises at least one agent selected from the group of aminoglycosides, β-lactam antibiotics, chloramphenicol, fluoroquinolones, glycopeptides, lincosamides, macrolides, polymixins, rifamycins, streptogramins, tetracyclines and diaminopyrimidines.

The term “antifungal drug” as used herein, refers to an agent or a composition with properties useful in the treatment against a fungi-related disease. An antifungal drug may have, inter alia, properties of preventing, inhibiting, suppressing, reducing, adversely impacting, and/or interfering with the growth, survival, replication, function, and/or dissemination of at least fungus.

In some embodiments, the antifungal drug described herein comprises at least one agent selected from the group of polyenes, azoles, allylamines, thiocarbamates, pyrimidine and benzimidazoles.

In some embodiments, the antifungal drug described herein comprises at least one agent selected from the group of griseofulvin, amphotericin b, natamycin, nystatin, clotrimazole, econazole, enilconazole, ketoconazole, miconazole, parconazole, fluconazole, itraconazole, terbinafine, tolnaftate, thiabendazole and flucytosine.

The term “antiparasitic drug” as used herein, refers to a drug with properties useful in the treatment against a parasite-related disease. An antiparasitic drug may have, inter alia, properties of preventing, inhibiting, suppressing, reducing, adversely impacting, and/or interfering with the growth, survival, replication, function, and/or dissemination of a parasite. In some embodiments, the antiparasitic drug described herein comprises at least one agent selected from the group of antinematodal, anticestodal, antilung worm, antitrematodal, antifilarial, antiprotozoal and insecticide.

In a further embodiment, the present invention relates to the anti-infective composition of the invention, wherein the pathogen is Streptococcus equi equi, Equine herpes virus, in particular Equine herpes virus 1 or 4.

In a further embodiment, the present invention relates to the anti-infective composition of the invention, wherein the pathogen is Equine influenza virus.

The method of the invention can efficiently determine the pathogen and facilitates early detection, screening, monitoring, and/or confirmation of a past infection.

The skilled person is aware how to treat an infection with a pathogen once the pathogen has been specified using the methods of the present invention. Therefore, the determination of the pathogen enabled by method of the invention can subsequently improve the treatment of an infection, reduce pathogen spreading and/or avoid disease progression.

In a further embodiment, the invention relates to a kit, in particular a kit for use in detecting a nucleic acid molecule in a sample, in particular detecting infection of an animal with a pathogen. The kit comprises one or more, preferably all five or six primers for detecting a pre-determined sequence of the pathogen. The kit may also comprise more than one primer system, in particular two or more primer systems targeting different sequences of the same pathogen or different pathogens, e.g., by using primers that contain a quencher-fluorophore duplex region (Tanner N A, Zhang Y, Evans T C Jr. Simultaneous multiple target detection in real-time loop-mediated isothermal amplification. Biotechniques. 2012; 53(2):81-89.). As such, the methods of the invention and the kit of the invention can be used to detect more than one different pathogen in a sample by using more than one primer system. In this regard, it was surprisingly found that the reduced number of primers leads to an improved usability of more than one primer systems due to reduced primer interference.

In a particularly preferred embodiment of the present invention, the kits (to be prepared in context) of this invention or the methods and uses of the invention may further comprise or be provided with (an) instruction manual(s). For example, said instruction manual(s) may guide the skilled person (how) to employ the kit of the invention in the diagnostic uses provided herein and in accordance with the present invention. Particularly, said instruction manual(s) may comprise guidance to use or apply the herein provided methods or uses.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The general methods and techniques described herein may be performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990).

While aspects of the invention are illustrated and described in detail in the figures and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below.

FIG. 1 shows a comparison of the five and six primer systems on an Equine influenza virus.

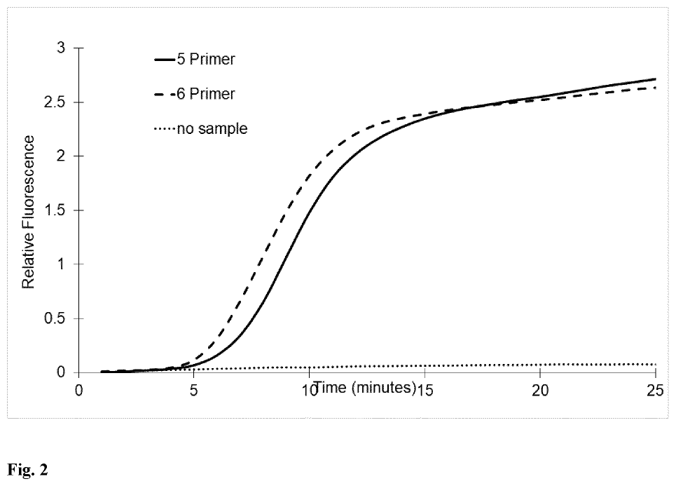

FIG. 2 shows a comparison of the five and six primer systems on Equine herpesvirus 1.

Furthermore, in the claims the word “comprising” does not exclude other elements or steps, and the indefinite article “a” or “an” does not exclude a plurality. A single unit may fulfill the functions of several features recited in the claims. The terms “essentially”, “about”, “approximately” and the like in connection with an attribute or a value particularly also define exactly the attribute or exactly the value, respectively. Any reference signs in the claims should not be construed as limiting the scope.

### EXAMPLES

The following are examples of methods and compositions of the invention. It is understood that various other embodiments may be practiced, given the general description provided above.

### The Novel 5 Primer System without F3 Amplifies Equine influenza Virus RNA as Efficient as the 6 Primer System with F3

TABLE 1
Primers

| | | | |
|---|---|---|---|
| FIP | CAA GTC TCT GCG CGA TCT AGG | LPF | TTT GAG GGG GCC TGA TGG |
| | TCG AAA CGT ACG TTC (SEQ ID | | (SEQ ID NO: 3) |
| | NO: 1) | | |
| BIP | GAA GAT GTC TTT GCA GGG TTG | LPB | ACC GAT CTT GAG GCA CTC |
| | GTC TTG TCT TTA GCC (SEQ ID | | (SEQ ID NO: 4) |
| | NO: 2) | | |
| B3 | AAT CCC TTT AGT YAG AG (SEQ | F3 | AGT CTT CTG ACC GAG GTC |
| | ID NO: 5) | | (SEQ ID NO: 6) |

TABLE 2
Primer mix: novel 5 primer system

| | Final concentration. |
|---|---|
| FIP | 1.6 μM |
| BIP | 1.6 μM |
| LPF | 0.8 μM |
| LPB | 0.8 μM |
| B3 | 0.4 μM |

TABLE 3
Primer mix: LAMP 6 primer system

| | Final concentration |
|---|---|
| FIP | 1.6 μM |
| BIP | 1.6 μM |
| LPF | 0.8 μM |
| LPB | 0.8 μM |
| B3 | 0.2 μM |
| F3 | 0.2 μM |

TABLE 4
Primer/Enzyme mix (PEM)

| | Vol/rx | |
|---|---|---|
| Isothermal master mix | 15.0 | μl |
| Primer mix | 2.0 | μl |
| | 17.0 | μl |

Add 17.0 μl PEM per reaction

### Template Addition

Add 8.0 μl of template RNA

Add 8.0 μl RNase-free H2O as negative assay control

TABLE 5
Settings for isothermal amplification and dye acquisition

| | Cycles | Temperature | Acquisition | Time | Ramp rate |
|---|---|---|---|---|---|
| Amplification | 25 | 65° C. | None | 27 s | 4.4° C. |
| | | | Single | 30 s | 4.4° C. |

| | | | Quant | Melt | Integration |
|---|---|---|---|---|---|
| | Channel | Dye | Factor | Factor | Time |
| Dye acquisition | #1,470/514 | SYBR | 20.00 | 1.2 | Dynamic |
| | | Green I | | | |

### The Novel 5 Primer System without F3 Amplifies Equine herpes Virus Type 1 DNA as Efficient as 6 Primer System with F3

TABLE 6
Primers

| | | | |
|---|---|---|---|
| FIP | GTC GTA RAA CCT GAG AGC | LPF | GAG CTG GTT GCG GCG CTG |
| | GGC CTG CTA GAC TAC AGC | | (SEQ ID NO: 9) |
| | (SEQ ID NO: 7) | | |
| BIP | CGA CAG CGT GGT CAA CGT | LPB | ATA CCG CAG TGA TTA TGC |
| | TGA AAA AGC TGG CGA TCC | | (SEQ ID NO: 10) |
| | (SEQ ID NO: 8) | | |
| B3 | TCC CCC ACT TTA CCC AG | F3 | CTG AGC TGG AGG ACA C |
| | (SEQ ID NO: 11) | | (SEQ ID NO: 12) |

TABLE 7
Primer mix: novel 5 primer system

| | Final concentration. |
|---|---|
| FIP | 1.6 μM |
| BIP | 1.6 μM |
| LPF | 0.8 μM |
| LPB | 0.8 μM |
| B3 | 0.4 μM |

TABLE 8
Primer mix: LAMP 6 primer system

| | Final concentration |
|---|---|
| FIP | 1.6 μM |
| BIP | 1.6 μM |
| LPF | 0.8 μM |
| LPB | 0.8 μM |
| B3 | 0.2 μM |
| F3 | 0.2 μM |

TABLE 9
Primer/Enzyme mix (PEM)

| | Vol/rx | |
|---|---|---|
| Isothermal master mix | 15.0 | μl |
| Primer mix | 2.0 | μl |
| | 17.0 | μl |

Add 17.0 μl PEM per reaction

### Template Addition

Add 8.0 μl template DNA

Add 8.0 μl RNase-free H2O as negative assay control

TABLE 10
Settings for isothermal amplification and dye acquisition

| | Cycles | Temperature | Acquisition | Time | Ramp rate |
|---|---|---|---|---|---|
| Amplification | 25 | 65° C. | None | 27 s | 4.4° C. |
| | | | Single | 30 s | 4.4° C. |

| | | | Quant | Melt | Integration |
|---|---|---|---|---|---|
| | Channel | Dye | Factor | Factor | Time |
| Dye acquisition | #1,470/514 | SYBR | 20.00 | 1.2 | Dynamic |
| | | Green I | | | |

1. A method for determining presence of a pre-determined nucleic acid sequence in a sample, the method comprising the steps of:
(a) adding one or more enzyme(s) providing activities of RNA- and/or DNA-dependent DNA polymerase activity and strand-displacement activity to the sample to be analysed for the presence of the pre-determined nucleic acid sequence;
(b) adding at least five DNA primers to the sample to be analysed for the presence of the pre-determined nucleic acid sequence, wherein at least one DNA primer comprises a sequence hybridisable to the nucleic acid sequence and at least one DNA primer comprises a sequence hybridisable to the DNA sequence reverse-complementary to the nucleic acid sequence;
(c) incubating the sample resulting from steps (a) and (b) at a fixed temperature;
(d) determining whether an elongated DNA sequence is present in the sample, wherein presence of the elongated DNA sequence in the sample is indicative of the presence of the pre-determined nucleic acid sequence in the sample
wherein the sample is obtained from an animal and wherein no F3 primer is used.

2. The method of claim 1, wherein four of the at least five primers are forward inner primer (FIP), backward inner primer (BIP), loop primer forward (LPF) and loop primer backwards (LPB), respectively.

3. The method of claims 1 and 2, wherein the fifth primer is a B3 primer.

4. The method of any one of claims 1 to 3, wherein the pre-determined nucleic acid sequence is an RNA or DNA sequence.

5. The method of any one of claims 1 to 4, wherein the pre-determined RNA or DNA sequence is comprised in a pathogen.

6. The method of claim 5, wherein the pathogen is a virus, a bacterium, a fungus or a parasite.

7. The method of embodiment 6, wherein the pathogen is Streptococcus equi equi, Equine herpes virus, in particular Equine herpesvirus 1 or 4, or Equine influenza virus

8. The method of any one of claims 1 to 7, wherein the fixed temperature is between 50 and 75° C.

9. The method of any one of claims 1 to 8, wherein the sample in step (c) is incubated for 1 to 120 minutes.

10. The method of any one of claims 1 to 9, wherein presence of the double-stranded elongated DNA sequence in the sample is determined by using a nucleic acid molecule hybridisable to the double-stranded elongated DNA sequence, in particular wherein the nucleic acid molecule is labelled, using a molecule that intercalates in the double-stranded elongated DNA sequence or using turbidity measurement.

11. A method of treating an animal infected by a pathogen, the method comprising administering to the animal an efficient amount of a therapeutic drug, wherein the animal has previously been determined to be infected by the pathogen using the method of any one of claims 1 to 10.

12. The method of claim 11 wherein the animal is a horse.

13. The method of claim 11 or 12, wherein the pathogen is a virus, a bacterium, a fungus or a parasite.

14. The method of any one of claim 11 or 13, wherein the therapeutic drug is an anti-viral, antibiotic, antifungal or antiparasitic drug, respectively.

15. The method of any one of claims 11 to 14, wherein the pathogen is Streptococcus equi equi, Equine herpes virus, in particular Equine herpesvirus 1 or 4, or Equine influenza virus.


SEQUENCE LISTING (14 sequences)

SEQ ID NO: 1 - DNA, 36 bp, Artificial Sequence
  FIP primer Equine influenza virus
caagtctctgcgcgatctaggtcgaaacgtacgttc

SEQ ID NO: 2 - DNA, 36 bp, Artificial Sequence
  BIP primer Equine influenza virus
gaagatgtctttgcagggttggtcttgtctttagcc

SEQ ID NO: 3 - DNA, 18 bp, Artificial Sequence
  LPF primer Equine influenza virus
tttgagggggcctgatgg

SEQ ID NO: 4 - DNA, 18 bp, Artificial Sequence
  LPB primer Equine influenza virus
accgatcttgaggcactc

SEQ ID NO: 5 - DNA, 17 bp, Artificial Sequence
  B3 primer Equine influenza virus
aatccctttagtyagag

SEQ ID NO: 6 - DNA, 18 bp, Artificial Sequence
  F3 primer Equine influenza virus
agtcttctgaccgaggtc

SEQ ID NO: 7 - DNA, 36 bp, Artificial Sequence
  FIP primer Equine herpes virus type 1
gtcgtaraacctgagagcggcctgctagactacagc

SEQ ID NO: 8 - DNA, 36 bp, Artificial Sequence
  BIP primer Equine herpes virus type 1
cgacagcgtggtcaacgttgaaaaagctggcgatcc

SEQ ID NO: 9 - DNA, 18 bp, Artificial Sequence
  LPF primer Equine herpes virus type 1
gagctggttgcggcgctg

SEQ ID NO: 10 - DNA, 18 bp, Artificial Sequence
  LPB primer Equine herpes virus type 1
ataccgcagtgattatgc

SEQ ID NO: 11 - DNA, 17 bp, Artificial Sequence
  B3 primer Equine herpes virus type 1
tcccccactttacccag

SEQ ID NO: 12 - DNA, 16 bp, Artificial Sequence
  F3 primer Equine herpes virus type 1
ctgagctggaggacac

SEQ ID NO: 13 - DNA, 982 bp, Artificial Sequence
  Influenza A virus (A/equine/Newmarket/11/2003(H3N8)) segment 7 matrix protein 2 (M2) and matrix protein 1 (M1) genes complete cds
atgagtcttctgaccgaggtcgaaacgtacgttctctctatcgtaccatcaggccccctcaaagccgagatcgcgcagagacttgaagatgtctttgcagggaagaacaccgatcttgaggcactcatggaatggctaaagacaagaccaatcctgtcacctttgactaaagggattttaggatttgtattcacgctcaccgtgcccagtgagcaaggactgcagcgtagacgctttgtccaaaatgcccttagtggaaacggagatccaaacaacatggacagagcagtaaaactgtacaggaagcttaaaagagaaataacattccatggggcaaaagaggtggcactaagctattccactggtgcactagccagctgcatgggactcatatacaacagaatgggaactgtgacaaccgaagtggcatttggcctggtatgcgccacatgtgaacagattgctgattcccagcatcgatctcacaggcagatggtgacaacaaccaacccattaatcagacatgaaaacagaatggtattagccagtaccacggctaaagccatggaacagatggcaggatcaagtgagcaggcagcagaggccatggaggttgctagtaaggctaggcagatggtacaggcaatgagaaccattgggacccaccctagctccagtgccggtttgaaagatgatctccttgaaaatttacaggcctaccagaaacggatgggagtgcaaatgcagcgattcaagtgatcctctcgttattgcagcaagtatcattgggatcttgcacttgatattgtggattcttgatcgtcttttcttcaaattcatttatcgtcgccttaaatacgggttgaaaagagggccttctacggaaggagtacctgagtctatgagggaagaatatcggcaggaacagcagaatgctgtggatgttgacgatggtcattttgtcaacatagagctggagtaa

SEQ ID NO: 14 - DNA, 974 bp, Artificial Sequence
  Equid herpesvirus 1 strain TR04 glycoprotein B (glyB)
gacaccacgcgaagacgcaggtctctcctgtcagtaccagaacctcagccaacccaagatggtgtgcatagagaacaaattctacatcgcttgcacaaacgagcagtggaggcaacggcaggtaccgattcttccaacgtcaccgccaaacagctggagctcatcaaaaccacgtcgtctatcgagtttgccatgctacagtttgcatacgatcacatccaatcccacgtcaatgaaatgctaagtagaatagcaactgcgtggtgtaccctccaaaacaaagagcggaccctatggaacgaaatggtgaagattaacccgagcgccatagtctccgcaacccttgacgagcgagttgcagcgagggtcctgggggacgtgatagctataacgcactgcgccaaaatagagggcaacgtgtacttgcaaaactccatgcgctcgatggacagtaacacgtgctactcccgcccccccgtaacatttacaattactaagaatgcaaacaacagagggtcgatagaaggccagctgggagaggagaacgagattttcacggagcgcaagctgatcgagccgtgcgccctcaatcagaagcgctactttaagtttggcaaagagtacgtttactacgagaactacacgttcgtccgcaaagtgccccccacggaaatcgaggttatcagcacgtacgttgaactaaacttgacccttttggaagaccgcgagtttctgcccctggaggtgtacacgcgggctgagctggaggacaccggcctgctagactacagcgaaatacagcgccgcaaccagctccacgctctcaggttttatgacatcgacagcgtggtcaacgtggacaataccgcagtgattatgcaggggatcgccagctttttcaagggcctgggtaaagtgggggaggccgtgggaacgctcgttctcggcgccgccggcgctgttgtttcaaccgt